(19) United States
(12) Patent Application Publication
(10) Pub. No.: US 2014/0081381 A1
(43) Pub. Date: Mar. 20, 2014

(54) POLYMERIC STENT

(71) Applicants: Hyung-Il Kim, Seoul (KR); Suntech Co., Seoul (KR)

(72) Inventor: Hyung-IL Kim, Seoul (KR)

(21) Appl. No.: 13/929,237

(22) Filed: Jun. 27, 2013

(30) Foreign Application Priority Data
Sep. 20, 2012 (KR) 10-2012-0104473

(51) Int. Cl. A61F 2/90

(52) U.S. Cl. CPC A61F 2/90
USPC 623/1.16; 29/428

(57) ABSTRACT

The present invention relates to a polymeric stent, and to an endoprosthesis that is implanted within blood vessels and is formed of a polymer.

### CROSS-REFERENCE TO RELATED APPLICATIONS

This application claims priority to and the benefit of Korean Patent Application No. 10-2012-0104473 filed in the Korean Intellectual Property Office on Sep. 20, 2012, the entire contents of which are incorporated herein by reference.

### TECHNICAL FIELD

The present invention relates to a polymeric stent, and to an endoprosthesis that is implanted within blood vessels and is formed of a polymer.

### BACKGROUND ART

In general, stents are expandable medical prostheses, and are used within body vessels of humans for a variety of medical applications.

Examples thereof include intravascular stents for treating stenoses, and stents for maintaining openings in the urinary, biliary, tracheobronchial, esophageal, and renal tracts, and inferior vena cava.

Typically, a delivery device that retains a stent in the compressed state is used to deliver the stent to a treatment site through body vessels.

In percutaneous transluminal angioplasty, an implantable endoprosthesis, that is, a stent is introduced through a delivery device, and is passed through vessel conduits to a treatment site. After the stent approaches the treatment site, the stent is mechanically expanded, usually with the aid of an inflatable balloon, thereby being expanded within the body vessel. The delivery device is then retreated and removed from the patient. The stent remains within the vessel at the treatment site as an implant,

### SUMMARY OF THE INVENTION

The present invention relates to a polymeric stent, and has been made in an effort to provide an endoprosthesis that is implanted within blood vessels and is formed of a polymer.

An exemplary embodiment of the present invention provides a polymeric stent including:

a repeating unit including a unit cell having a shape of a closed figure that looks like a V-letter and including a first hinge portion bent inwardly to the unit cell and a second hinge portion facing the first hinge portion and bent outwardly from the unit cell; and a linker portion extended outwardly from a bent portion of the second hinge portion

in which the repeating units are disposed such that an end of a linker portion of one repeating unit is connected to a bent portion of a first hinge portion of another adjacent repeating unit, or

an end of a first hinge portion and an end of a second hinge portion of one repeating unit are connected to an end of a first hinge portion and an end of a second hinge portion of the another adjacent repeating unit, respectively.

Another exemplary embodiment of the present invention provides a method for manufacturing a polymeric stent, including: forming a repeating unit including a unit cell having a shape of a closed figure that looks like a V-letter and including a first hinge portion bent inwardly to the unit cell and a second hinge portion facing the first hinge portion and bent outwardly from the unit cell; and a linker portion extended outwardly from a bent portion of the second hinge portion; and

disposing the repeating units, such that an end of a linker portion of one repeating unit is connected to a bent portion of a first hinge portion of another adjacent repeating unit, or

forming the repeating units continuously such that an end of a first hinge portion and an end of a second hinge portion of one repeating unit are connected to an end of a first hinge portion and an end of a second hinge portion of another adjacent repeating unit, respectively.

The polymeric stent according to an exemplary embodiment of the present invention is advantageous in that the mechanical strength thereof is high.

The polymeric stent according to an exemplary embodiment of the present invention is easily handled when inserted into body vessels because the strut thereof is thin.

The polymeric stent according to an exemplary embodiment of the present invention is advantageous in that the form thereof is slightly distorted by resistance when the polymeric stent is implanted into blood vessels.

The polymeric stent according to an exemplary embodiment of the present invention is advantageous in that the patterns thereof are not entangled with each other when the polymeric stent is expanded.

The polymeric stent according to an exemplary embodiment of the present invention is advantageous in that the polymeric stent has good flexibility because the strut thereof is relatively thin.

### BRIEF DESCRIPTION OF THE DRAWINGS

FIG. 1 is a schematic view of a pattern of a polymeric stent according to an exemplary embodiment of the present invention.

FIG. 2 is an enlarged view of FIG. 1.

FIG. 3 illustrates the shape of a pattern of the polymeric stent according to an exemplary embodiment of the present invention in each state of (a) when the polymeric stent is crimped, or (b) when the polymeric stent is expanded, respectively.

FIG. 4 illustrates the change in shape of the unit cell according to the interior angle of the second hinge portion of the polymeric stent according to an exemplary embodiment of the present invention.

FIG. 5 illustrates the change in pattern according to the thickness of the strut of the polymeric stent according to an exemplary embodiment of the present invention.

### DETAILED DESCRIPTION

The polymeric stent of the present invention includes: a repeating unit including a unit cell having a shape of a closed figure that looks like a V-letter and including a first hinge portion bent inwardly to the unit cell and a second hinge portion facing the first hinge portion and bent outwardly from the unit cell;

a linker portion extended outwardly from a bent portion of the second hinge portion.

In an exemplary embodiment of the present invention, the repeating units may be disposed such that an end of a linker portion of one repeating unit is connected to a bent portion of a first hinge portion of another adjacent repeating unit, or

an end of a first hinge portion and an end of a second hinge portion of one repeating unit may be disposed so as to be connected to an end of a first hinge portion and an end of a second hinge portion of the adjacent, other repeating unit, respectively.

The polymeric stent according to an exemplary embodiment of the present invention includes three or more repeating units,

the repeating units may be disposed such that an end of a linker portion of one repeating unit is connected to a bent portion of a first hinge portion of another adjacent repeating unit, and

an end of a first hinge portion and an end of a second hinge portion of one repeating unit may be disposed so as to be connected to an end of a first hinge portion and an end of a second hinge portion of another adjacent repeating unit, respectively.

The polymeric stent of the present invention may be manufactured by disposing one or more of the repeating unit in a row.

In an exemplary embodiment of the present invention, when the polymeric stent is a tube type, 4 to 15 repeating units may be disposed in a circumferential direction of the polymeric stent.

The polymeric stent according to an exemplary embodiment of the present invention may be formed in the form of a cylinder by connecting one end of the pattern of the polymeric stent manufactured by repeating the repeating unit of the present invention to the other end thereof.

In an exemplary embodiment of the present invention, when an end of a first hinge portion and an end of a second hinge portion of one repeating unit are disposed so as to be connected to an end of a first hinge portion and an end of a second hinge portion of another adjacent repeating unit, respectively, a line in which one or more unit cells are consecutively disposed side by side and a line in which one or more linker portions among the repeating units are disposed spaced apart from each other are formed.

At this time, the line of the unit cell is defined as the (A) row, and the line of the linker portion is defined as the (B) row.

In an exemplary embodiment of the present invention, when the repeating unit of the present invention is repeatedly disposed in one direction and in the other direction vertical to the one direction, a closed figure may be formed even at the (B) row which is the line of the linker portion.

In an exemplary embodiment of the present invention, when the repeating unit of the present invention is repeatedly disposed in one direction and in the other direction vertical to the one direction, a closed figure of a V-letter type shape, which is positioned in a direction opposite to a unit cell having a shape of a closed figure that looks like a V-letter at the (A) row, may be formed at the (B) row which is a line of the linker portion.

In an exemplary embodiment of the present invention, when one end (a) of the pattern of the polymeric stent is connected to the other end (b) thereof, open figures at both ends of the (B) row may be connected to each other to form a closed figure.

In an exemplary embodiment of the present invention, when one end (a) of the pattern of the polymeric stent is connected to the other end (b) thereof, open figures at both ends of the (B) row may be connected to each other to form a closed figure having a V-letter type shape, which is positioned in a direction opposite to the unit cell having a shape of a closed figure that looks like a V-letter at the (A) row thereof.

In the repeating unit of an exemplary embodiment of the present invention, the length of the linker portion may be shorter than the distance between the bent portion of the first hinge portion and the bent portion of the second hinge portion.

Accordingly, the unit cell having a shape of a closed figure that looks like a V-letter at the (A) row may be larger than a closed figure having a V-letter type shape at the (B) row, which is formed by the linker portion. In addition, in a longitudinal direction of the linker portion, the height of the unit cell having a shape of a closed figure that looks like a V-letter at the (A) row may be larger than the height of a closed figure having a V-letter type shape at the (B) row, which is formed by the linker portion.

In the repeating unit of an exemplary embodiment of the present invention, the length of the linker portion may be longer than the distance between the bent portion of the first hinge portion and the bent portion of the second hinge portion.

Accordingly, the unit cell having a shape of a closed figure that looks like a V-letter at the (A) row may be smaller than a closed figure having a V-letter type shape at the (B) row, which is formed by the linker portion. Furthermore, in a longitudinal direction of the linker portion, the height of the unit cell having a shape of a closed figure that looks like a V-letter at the (A) row may be smaller than the height of a closed figure having a V-letter type shape at the (B) row, which is formed by the linker portion.

In the polymeric stent according to an exemplary embodiment of the present invention, a corner and an end of one repeating unit are connected to a corner and an end of another unit cell. That is, in the polymeric stent according to an exemplary embodiment of the present invention, a repeating unit does not have a corner or end which is not connected to each other.

The polymeric stent of the present invention does not have a corner or end which is net connected to each other, and thus is advantageous in that the polymeric stent of the present invention has a relatively higher mechanical strength than that of a polymeric stent that is formed of a strut having the same thickness.

The corner or end which is not connected to each other in a polymeric stent fails to serve to support the polymeric stent, and thus the polymeric stent of the present invention, which does not have a corner or end which is not connected to each other, has a relatively high mechanical, strength.

In the same thickness, the polymeric stent has a relatively lower mechanical strength than that of a metal stent, and thus a strut of the polymeric stent is manufactured with a thickness larger than that of a strut of the metal stent.

The corner or end which is not connected to each other in the polymeric stent fails to serve to support the polymeric stent, and thus a strut is manufactured thicker than usual in order to impart a mechanical strength with a predetermined value or more.

However, the polymeric stent of the present invention does not have a corner or end which is not connected to each other, and thus is advantageous in that a mechanical strength with a predetermined value or more may be imparted with a relatively thin strut.

The polymeric stent of the present invention may be manufactured with a relatively thin strut, and thus is advantageous in that the polymeric stent has good flexibility.

In the polymeric stent according to an exemplary embodiment of the present invention, the thinner the thickness of the strut is manufactured, the smaller the size of the repeating unit becomes, thereby increasing the number of repeating units per the same area. Through this, the mechanical strength of the polymeric stent may be maintained.

Further, the polymeric stent in the related art is disadvantageous in that after the polymeric stent is implanted into the blood vessels, the corner which is not connected to each other may hamper the blood stream within the blood vessels and forms hematoma and the like. However, the polymeric stent of the present invention does not have a corner or end which is not connected to each other, and thus the aforementioned problem may be alleviated.

In the present invention, the corner means a point that is positioned at an end and a bent portion of the first hinge portion, an end and a bent portion of the second hinge portion, and an end of the linker portion in one repeating unit.

In an exemplary embodiment of the present invention, the interior angle of the second hinge portion may be 100 degrees or more.

In an exemplary embodiment of the present invention, the interior angle of the second hinge portion may be 90 to 160 degrees.

In an exemplary embodiment of the present invention, in the repeating unit, the area of a rectangle that passes through both ends of the first hinge portion, both ends of the second hinge portion and an end of the linker portion may be 0.1 to 45 mm² and 0.13 to 44.38 mm², if necessary. At this time, the rectangle includes rectangles and squares.

In an exemplary embodiment of the present invention, the repeating unit may include a strut of the unit cell, which is a line that forms the closed figure having a V-letter type shape of the unit cell and a strut of the linker portion, which is a line that forms the linker portion.

In an exemplary embodiment of the present invention, the repeating unit includes a strut of the unit cell, which is a line that forms the closed figure having a V-letter type shape of the unit cell and a strut of the linker portion, which is a line that forms the linker portion, and

in the polymeric stent, the area of the strut of the unit cell and the strut of the linker portion in the repeating unit may be 5 to 46%, based on the area of the rectangle which passes through both ends of the first hinge portion, both ends of the second hinge portion, and an end of the linker portion.

If necessary, the area of the strut of the unit cell and the strut of the linker portion in the repeating unit may be 15 to 40% and 25 to 35%, based on the area of the rectangle which passes through both ends of the first hinge portion, both ends of the second hinge portion, and an end of the linker portion.

At this time, in the repeating unit, the area of the rectangle that passes through both ends of the first hinge portion, both ends of the second hinge portion and an end of the linker portion means an area of a region that one unit cell and a linker portion thereof occupy, and the area of the strut of one unit cell and the strut of the linker portion means an area that the line of each strut occupies.

In an exemplary embodiment of the present invention, the thickness of the strut of the unit cell and the strut of the linker portion may be 80 to 160 μm.

At this time, the thickness of the strut means a length thereof in a direction that is vertical to the longitudinal direction. That is, the thickness of the strut means the line width of the strut.

In an exemplary embodiment of the present invention, when the polymeric stent is a tube type, the diameter of the polymeric stent may be 2 to 8 mm.

In the present invention, when the polymeric stent is a tube type, in a cross-section which is a direction that is vertical to the axis, the diameter of the polymeric stent means a length of a line segment that connects two points on the cross-section so as to pass through the center of the cross-section.

At this time, the diameter of the polymeric stent means a diameter of a polymeric stent formed by connecting one end of the pattern of the polymeric stent with the other end thereof.

In an exemplary embodiment of the present invention, the polymeric stent may include a biodegradable polymer.

The biodegradable polymer is not particularly limited as long as the polymer may be degraded in vivo.

For example, the biodegradable polymer may be a synthetic biodegradable polymer or natural biodegradable polymer.

The synthetic biodegradable polymer may be one polymer selected from polyglycolide, polylactide, poly p-dioxanone, polycaprolactobne, trimethylene carbonate, polyhydroxyal kanoates, polypropylene fumarate, polyortho esters, other polyester, polyanhydride, polyphosphazenes, polyalkylcyanoacrylate, poloxamers, and polyamino L-tyrosine, or a copolymer thereof or a mixture thereof.

In addition, the natural biodegradable polymer may be a material selected from modified polysaccharides, oxidized cellulose, gelatin, and collagen, or a mixture of one or more thereof.

The stent of the present invention is an expandable medical prosthetic device, is implanted within body vessels of humans for a variety of medical applications, and serves to maintain the form of the body vessel.

The stent of the present invention may be used as intravascular stents for treating stenoses, stents for maintaining openings in the urinary, biliary, tracheobronchial, esophageal, and renal tracts, and inferior vena cava, and the like.

The stent of the present invention is delivered to a treatment site through various body vessels by using a delivery device. After the stent is positioned at the treatment site, the delivery device is actuated to release the stent and the stent is mechanically expanded, usually with the aid of an inflatable balloon, within the body vessel. The delivery device is then detached from the stent, and the stent remains within the body vessel at the treatment site as an implant.

The polymeric stent according to an exemplary embodiment of the present invention is advantageous in that the form thereof is less distorted by resistance when the polymeric stent is implanted into blood vessels.

The polymeric stent according to an exemplary embodiment of the present invention is advantageous in that the patterns thereof are not entangled with each other when the polymeric stent is expanded.

When the polymeric stent of the present invention is manufactured of a biodegradable polymer, the stent remains within the body vessel at the treatment site, is naturally degraded after a certain time, and thus may disappear.

At this time, time for the polymeric stent of the present invention to be degraded may be about 2 years.

In the polymeric stent according to an exemplary embodiment of the present invention, the repeating unit may be formed by processing a polymeric tube using laser. Specifically, the repeating unit of the present invention may be formed on the side surface of the polymeric tube.

The present invention provides a method for manufacturing a polymeric stent, the method including: forming a repeating unit including a unit cell having a shape of a closed figure that looks like a V-letter and including a first hinge portion bent inwardly to the unit cell and a second hinge portion facing the first hinge portion and bent outwardly from the unit cell; and a linker portion extended outwardly from a bent portion of the second binge portion; and

disposing the repeating units, such that an end of a linker portion of one repeating unit is connected to a bent portion of a first hinge portion of another adjacent repeating unit, or

forming the repeating units continuously such that an end of a first hinge portion and an end of a second hinge portion of one repeating unit are connected to an end of a first hinge portion and an end of a second hinge portion of another adjacent repeating unit, respectively.

In an exemplary embodiment of the present invention, the repeating unit may be formed by using laser.

In an exemplary embodiment of the present invention, the repeating unit may be formed on the side surface of the polymeric tube.

In the present invention, the tube means one having a long axial length.

Hereinafter, preferred embodiments of the resent invention will be described in more detail with reference to the accompanying drawings.

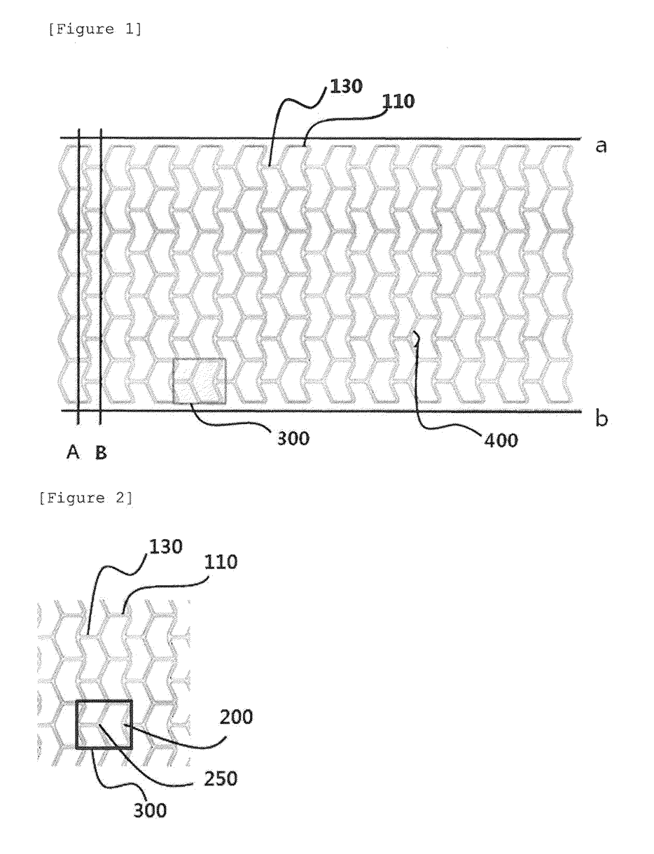

FIG. 1 of the present invention is a schematic view of the pattern of the polymeric stent according to an exemplary embodiment of the present invention, and FIG. 2 is an enlarged view of FIG. 1.

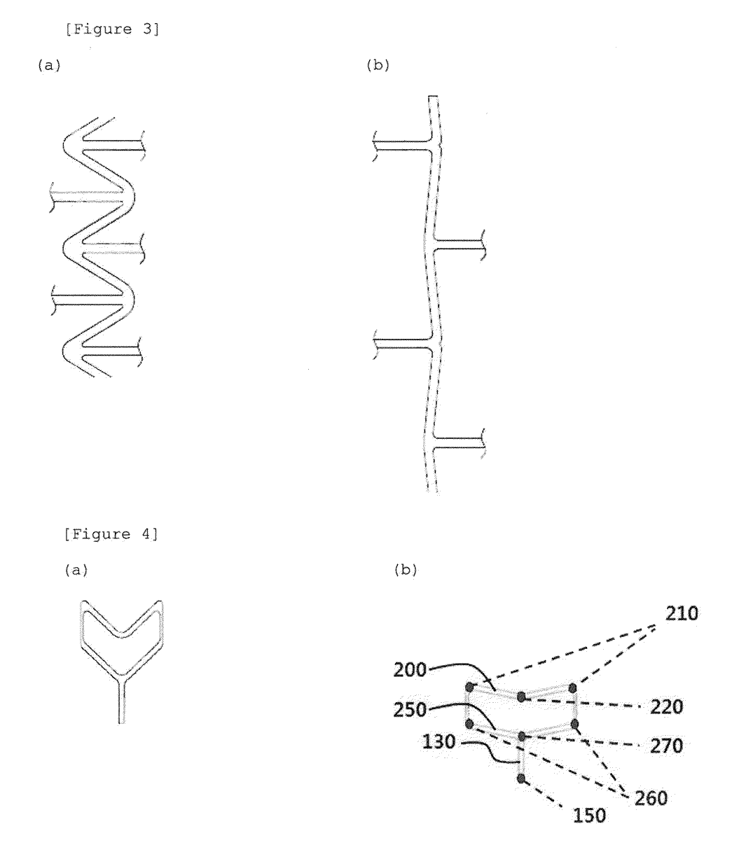

FIG. 3 of the present invention illustrates the shape of the pattern of the polymeric stent according to an exemplary embodiment of the present invention in each state of (a) when the polymeric stent is crimped, or (b) when the polymeric stent is expanded, respectively.

FIG. 4 of the present invention illustrates the change in shape of the unit cell according to the interior angle of the second hinge portion of the polymeric stent according to an exemplary embodiment of the present invention.

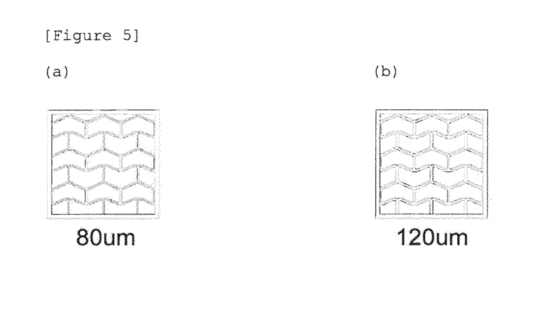

FIG. 5 of the present invention illustrates the change in pattern according to the thickness of the strut of the polymeric stent according to an exemplary embodiment of the present invention.

As illustrated in FIGS. 1 and 2, the polymeric stent of the present invention includes a unit cell 110 having a shape of a close figure that looks like a V-letter and including a first hinge portion 200 bent inwardly to the unit cell 110 and a second hinge portion 250 facing the first hinge portion 200 and bent outwardly from the unit cell; and

a repeating unit 300 including a linker portion 130 extended outwardly from a bent portion of the second hinge portion.

In an exemplary embodiment of the present invention, as illustrated in FIGS. 1 and 2, the repeating units 300 may be disposed such that an end of a linker portion of one repeating unit is connected to a bent portion of a first hinge portion of another adjacent repeating unit, or

an end of a first hinge portion and an end of a second hinge portion of one repeating unit are connected to an end of a first hinge portion and an end of a second hinge portion of another adjacent repeating unit, respectively.

The polymeric stent according to an exemplary embodiment of the present invention includes three or more repeating units,

the repeating units may be disposed such that an end of a linker portion of one repeating unit is connected to a bent portion of a first hinge portion of another adjacent repeating unit, and

an end of a first hinge portion and an end of a second hinge portion of one repeating unit are connected to an end of a first hinge portion and an end of a second hinge portion of further another adjacent repeating unit, respectively.

As illustrated in FIGS. 1 and 2, the polymeric stent of the present invention may be manufactured by disposing one or more of the repeating unit 300 in a row.

In an exemplary embodiment of the present invention, when the polymeric stent is a tube type, 4 to 15 repeating units may be disposed in a circumferential direction of the polymeric stent.

As illustrated in FIG. 1, the polymeric stent according to an exemplary embodiment of the present invention may be formed in the form of a cylinder by connecting one end (a) of the pattern of the polymeric stent manufactured by repeating the repeating unit 300 of the present invention to the other end (b) thereof. In an exemplary embodiment of the present invention, as illustrated in FIG. 1, a line disposed such that an end of a first hinge portion and an end of a second hinge portion of one unit cell are connected to an end of a first hinge portion and an end of a second hinge portion of another adjacent unit cell, respectively, is defined as the (A) row.

Furthermore, a line of the linker portion 130 formed according to the disposition of the unit cell is defined as the (B) row.

In an exemplary embodiment of the present invention, as illustrated in FIG. 1, when the repeating unit 300 of the present invention is repeatedly disposed in one direction and in the other direction vertical to the one direction, a closed figure may be formed even at the (B) row which is the line of the linker portion.

In an exemplary embodiment of the present invention, as illustrated in FIG. 1, when the repeating unit 300 of the present invention is repeatedly disposed in one direction and in the other direction vertical to the one direction, a closed figure having a V-letter type shape, which is positioned in a direction opposite to the unit cell having a shape of a closed figure that looks like a V-letter may be formed at the (B) row which is a line of the linker portion.

In an exemplary embodiment of the present invention, as illustrated in FIG. 1, when one end (a) of the pattern of the polymeric stent is connected to the other end (b) thereof, open figures at both ends of the (B) row may be connected to each other to form a closed figure.

In an exemplary embodiment of the present invention, as illustrated in FIG. 1, when one end (a) of the pattern of the polymeric stent is connected to the other end (b) thereof, open figures at both ends of the (B) row may be connected to each other to form a closed figure having a V-letter type shape, which is positioned in a direction opposite to the unit cell having a shape of a closed figure that looks like a V-letter at the (A) row thereof.

In the repeating unit 300 in an exemplary embodiment of the present invention, the length of the linker portion 130 may be shorter than the distance between the bent portion of the first hinge portion 200 and the bent portion of the second hinge portion 250.

Accordingly, in FIG. 1, the unit cell having a shape of a closed figure that looks like a V-letter at the (A) row may be larger than a closed figure having a V-letter type shape at the (B) row, which is formed by the linker portion. Further, in a longitudinal direction of the linker portion 130, the height of the unit cell having a shape of a closed figure that looks like a V-letter at the (A) row may be larger than the height of a closed figure having a V-letter type shape at the (B) row, which is formed by the linker portion.

In the repeating unit 300 in an exemplary embodiment of the present invention, the length of the linker portion 130 may be longer than the distance between the bent portion of the first hinge portion 200 and the bent portion of the second hinge portion 250.

Accordingly, in FIG. 1, the unit cell having a shape of a closed figure that looks like a V-letter at the (A) row may be smaller than a closed end having a V-letter type shape at the (B) row, which is formed by the linker portion. In addition, in a longitudinal direction of the linker portion 130, the height of the unit cell having a shape of a close figure that looks like a V-letter at the (A) row may be smaller than the height of a closed figure having a V-letter type shape at the (B) row, which is formed by the linker portion.

In the polymeric stent according to an exemplary embodiment of the present invention, as illustrated in FIGS. 1 and 2, a corner and an end of one repeating unit 300 are connected to a corner and an end of another unit cell. That is, in the polymeric stent according to an exemplary embodiment of the present invention, the repeating unit 300 does not have a corner or an end which is not connected to each other.

The polymeric stent of the present invention has no corner or end which is not connected to each other, and thus is advantageous in that the polymeric stent of the present invention has a relatively higher mechanical strength than that of a polymeric stent that is formed of a strut having the same thickness.

The corner or end which is not connected to each other in the polymeric stent fails to serve to support the polymeric stent, and thus the polymeric stent of the present invention, which does not have a corner or end which is not connected to each other, has a relatively high mechanical strength.

In the same thickness, the polymeric stent has a lower mechanical strength than that of a metal stent, and thus a strut of the polymeric stent is manufactured with a thickness larger than that of a strut of the metal stent.

The corner or end which is not connected to each other in the polymeric stent fails to serve to support the polymeric stent, and thus a strut is manufactured thicker than usual in order to impart a mechanical strength with a predetermined value or more.

However, the polymeric stent of the present invention does not have a corner or end which is not connected to each other, and thus is advantageous in that a mechanical strength with a predetermined value or more may be imparted with a relatively thin strut.

The polymeric stent of the present invention may be manufactured with a relatively thin strut, and thus is advantageous in that the polymeric stent has good flexibility.

In the polymeric stent according to an exemplary embodiment of the present invention, the thinner the thickness of the strut is manufactured, the smaller the size of the repeating unit becomes, thereby increasing the number of repeating units per the same area. Through this, the mechanical strength of the polymeric stent may be maintained.

Further, the polymeric stent in the related art is disadvantageous in that after the polymeric stent is implanted into blood vessels, the corner which is not connected to each other may hamper the blood stream within the blood vessels and forms hematoma and the like. However, the polymeric stent of the present invention does not have a corner or end which is not connected to each other, and thus the aforementioned problem may be alleviated.

In the present invention, the corner means a point that is positioned at an end and a bent portion of the first hinge portion, an end and a bent portion of the second hinge portion, and an end of the linker portion in one repeating unit.

In an exemplary embodiment of the present invention, the interior angle 400 of the second hinge portion may be 100 degrees or more.

In an exemplary embodiment of the present invention, the interior angle 400 of the second hinge portion may be 90 to 160 degrees or more.

FIG. 4 illustrates (a) a form of the unit cell when the interior angle 400 of the second hinge portion is 90 degrees and (b) a form of the unit cell when the interior angle 400 of the second hinge portion is 160 degrees.

In an exemplary embodiment of the present invention, in the repeating unit, the area of a rectangle that passes through both ends of the first hinge portion, both ends of the second hinge portion and an end of the linker portion may be 0.1 to 45 mm² and 0.13 to 44.38 mm², if necessary. At this time, the area of the rectangle means an area of a region that one unit cell and a linker portion thereof occupy. That is, the area of the rectangle means an area of the rectangle which is a region that one unit cell and a linker portion thereof, which are illustrated in FIGS. 1 and 2, occupy.

In an exemplary embodiment of the present invention, the repeating unit 300 includes a strut of the unit cell, which is a line that forms the closed figure that looks like a V-letter of the unit cell and a strut of the linker portion, which is a line that forms the linker portion, and

in the polymeric stent, the area of the strut of the unit cell and the strut of the linker portion in the repeating unit may be 5 to 46%, based on the area of the rectangle which passes through both ends of the first hinge portion, both ends of the second hinge portion, and an end of the linker portion.

If necessary, the area of the strut of the unit cell and the strut of the linker portion in the repeating unit may be 15 to 40% and 25 to 35%, based on the area of the rectangle which passes through both ends of the first hinge portion, both ends of the second hinge portion, and an end of the linker portion.

At this time, the area of the rectangle means an area of a region that one unit cell 110 and a linker portion 130 thereof occupy, and the area of the strut of one unit cell and the strut of the linker portion means an area that the line of each strut occupies.

In an exemplary embodiment of the present invention, the thickness of the strut of the unit cell and the strut of the linker portion may be 80 to 160 μm.

At this time, the thickness of the strut means a length thereof in a direction that is vertical to the longitudinal direction. That is, the thickness of the strut means the line width of the strut.

FIG. 5 illustrates (a) the pattern of the polymeric stent when the thickness of the unit cell 110 and the linker portion 130 is 80 μm and (b) the pattern of the polymeric stent when the thickness of the unit cell 110 and the linker portion 130 is 120 μm.

In an exemplary embodiment of the present invention, when the polymeric stent is a tube type, the diameter of the polymeric stent may be 2 to 8 mm.

In the present invention, when the polymeric stent is a tube type, in a cross-section in a direction that is vertical to the axis, the diameter of the polymeric stent means a length of a line segment that connects two points on the cross-section so as to pass through the center of the cross-section.

At this time, the diameter of the polymeric stent means a diameter of a polymeric stent formed by connecting an end a on one side of the pattern of the polymeric stent with an end b on the other side thereof in FIG. 1.

In an exemplary embodiment of the present invention, the polymeric stent may include a biodegradable polymer.

The biodegradable polymer is not particularly limited as long as the polymer may be degraded in vivo.

For example, the biodegradable polymer may be a synthetic biodegradable polymer or natural biodegradable polymer.

The synthetic biodegradable polymer may be one polymer selected from polyglycolide, polylactide, poly p-dioxanone, polycaprolactone, trimethylene carbonate, polyhydroxyalkanoates, polypropylene fumarate, polyortho esters, other polyester, polyanhydride, polyphosphazenes, polyalkylcyanoacrylate, poloxamers, and polyamino L-tyrosine, or a copolymer thereof or a mixture thereof.

In addition, the natural biodegradable polymer may be a material selected from modified polysaccharides, oxidized cellulose, gelatin, and collagen, or a mixture of one or more thereof.

The stent of the present invention is an expandable medical prosthetic device, is implanted within the body vessels of humans for a variety of medical applications, and serves to maintain the form of the body vessel.

The stent of the present invention may be used as intravascular stents for treating stenoses, stents for maintaining openings in the urinary, biliary, tracheobronchial, esophageal, and renal tracts, and inferior vena cava, and the like.

The stent of the present invention is delivered to a treatment site through various body vessels by using a delivery device. After the stent is positioned at the treatment site, the delivery device is actuated to release the stent and the stent is mechanically expanded, usually with the aid of an inflatable balloon, within the body vessel. The delivery device is then detached from the stent, and the stent remains within the body vessel at the treatment site as an implant.

The polymeric stent according to an exemplary embodiment of the present invention is advantageous in that the form thereof is slightly distorted by resistance when the polymeric stent is implanted into blood vessels.

The polymeric stent according to an exemplary embodiment of the present invention is advantageous in that the patterns thereof are not entangled with each other when the polymeric stent is expanded.

When the polymeric stent of the present invention is manufactured of a biodegradable polymer, the stent remains within the body vessel at the treatment site, is naturally degraded after a certain time, and thus may disappear.

At this time, time for the polymeric stent of the present invention to be degraded may be about 2 years.

In the polymeric stent according to an exemplary embodiment of the present invention, the repeating unit may be formed by processing a polymeric tube using laser. Specifically, the repeating unit of the present invention may be formed on the side surface of the polymeric tube.

Hereinafter, the present specification will be described in detail with respect to Examples. The following Examples are for illustrating the present invention, and the scope of the present invention includes the scope described in the following claims and the substitutions and modifications thereof, and is not limited to the scope of Examples.

In the polymeric stent of the present invention, when the length of the diameter, the number of patterns in a circumference and the thickness of the strut are modified, the interior angle of the second hinge portion, the area of the repeating unit, and the variations of the area ratio of the strut based on the area of the repeating unit are shown in the following Table 1.

TABLE 1

| Diameter length (mm) | | | | | | | |
|---|---|---|---|---|---|---|---|
| | Number | Thickness | | | | | |
| | of cells | (um) | 2 mm | 3 mm | 5 mm | 6 mm | 8 mm |
| Area of | 4 ea | 80 | 2.57 | 6.31 | 17.9 | 25.05 | 44.38 |
| minimum | | 120 | 2.43 | 6.12 | 17.6 | 24.86 | 43.88 |
| unit | | 160 | 2.32 | 6.11 | 17.58 | 24.67 | 43.38 |
| cell | 9 ea | 80 | 0.45 | 1.15 | 3.39 | 4.74 | 8.52 |
| (mm²) | | 120 | 0.39 | 1.06 | 3.22 | 4.77 | 8.29 |
| | | 160 | 0.36 | 1.07 | 3.32 | 4.74 | 8.07 |
| | 15 ea | 80 | 0.13 | 0.35 | 1.11 | 1.59 | 3.21 |
| | | 120 | — | 0.30 | 1.01 | 1.49 | 2.79 |
| | | 160 | — | — | 0.92 | 1.39 | 2.66 |
| Area | 4 ea | 80 | 17 | 10 | 6 | 5 | 4 |
| ratio | | 120 | 26 | 16 | 10 | 8 | 6 |
| (%) | | 160 | 35 | 21 | 12 | 12 | 18 |
| | 9 ea | 80 | 38 | 24 | 14 | 13 | 9 |
| | | 120 | 58 | 37 | 22 | 18 | 14 |
| | | 160 | 74 | 46 | 27 | 24 | 19 |
| | 15 ea | 80 | 65 | 43 | 25 | 21 | 13 |
| | | 120 | — | 64 | 38 | 32 | 24 |
| | | 160 | — | — | 52 | 43 | 32 |
| V strut | 4 ea | 80 | 1.69 | 2.59 | 4.37 | 5.23 | 6.40 |
| length | | 120 | 1.72 | 2.63 | 4.38 | 5.19 | 6.46 |
| (mm) | | 160 | 1.76 | 2.60 | 4.38 | 5.18 | 6.80 |
| | 9 ea | 80 | 0.79 | 1.18 | 1.94 | 2.32 | 2.94 |
| | | 120 | 0.85 | 1.16 | 1.95 | 2.31 | 2.84 |
| | | 160 | 0.91 | 1.16 | 1.92 | 2.30 | 3.01 |
| | 15 ea | 80 | 0.48 | 0.72 | 1015 | 1.3 | 1.74 |
| | | 120 | — | 0.71 | 1.19 | 1.34 | 1.83 |
| | | 160 | — | — | 1.05 | 1.42 | 1.86 |

In the polymeric stent of the present invention, when the length of the diameter, the number of patterns in a circumference and the thickness of the strut are modified, the variations in the form of the unit cell are shown in the following Table 2.

| Diameter length (mm) | | | | | | |
|---|---|---|---|---|---|---|
| Number of | Thickness | | | | | |
| cells | (um) | 2 mm | 3 mm | 5 mm | 6 mm | 8 mm |
| 4 ea | 80 | [symbol] | [symbol] | 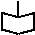 | 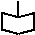 | 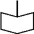 |
| | 120 | | [symbol] | 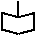 | 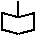 | 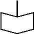 |
| | 160 | | | 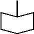 | 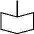 | 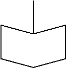 |
| 9 ea | 80 | [symbol] | [symbol] | [symbol] | [symbol] | 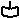 |
| | 120 | [symbol] | [symbol] | [symbol] | [symbol] | 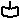 |
| | 160 | [symbol] | [symbol] | [symbol] | 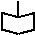 | 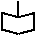 |
| 15 ea | 80 | [symbol] | [symbol] | [symbol] | [symbol] | [symbol] |
| | 120 | [symbol] | [symbol] | [symbol] | [symbol] | [symbol] |
| | 160 | [symbol] | [symbol] | [symbol] | [symbol] | 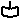 |

[symbol]

indicates data missing or illegible when filed

1. A polymeric stent, comprising:
a repeating unit comprising a unit cell having a shape of a closed figure that looks like a V-letter and comprising a first hinge portion bent inwardly to the unit cell and a second hinge portion facing the first hinge portion and bent outwardly from the unit cell; and a linker portion extended outwardly from a bent portion of the second hinge portion,
wherein the repeating units are disposed such that an end of a linker portion of one repeating unit is connected to a bent portion of a first hinge portion of another adjacent repeating unit, or
an end of a first hinge portion and an end of a second hinge portion of one repeating unit are connected to an end of a first hinge portion and an end of a second hinge portion of the another adjacent repeating unit, respectively.

2. The polymeric stent of claim 1, wherein the polymeric stent comprises three or more repeating units,
the repeating units are disposed such that an end of a linker portion of one repeating unit is connected to a bent portion of a first hinge portion of another adjacent repeating unit, and
an end of a first hinge portion and an end of a second hinge portion of one repeating unit are connected to an end of a first hinge portion and an end of a second hinge portion of further another adjacent repeating unit, respectively.

3. The polymeric stent of claim 1, wherein in the polymeric stent, all corners and ends of one repeating unit are connected to corners and ends of another adjacent repeating unit.

4. The polymeric stent of claim 1, wherein when the polymeric stent is a tube type, 4 to 15 repeating units are disposed in a circumferential direction of the polymeric stent.

5. The polymeric stent of claim 1, wherein an interior angle of the second hinge portion is 100 degrees or more.

6. The polymeric stent of claim 1, wherein in the repeating unit, an area of a rectangle that passes through both ends of the first hinge portion, both ends of the second hinge portion and an end of the linker portion is 0.1 to 45 mm².

7. The polymeric stent of claim 1, wherein the repeating unit comprises a strut of the unit cell, which is a line that forms the shape of a closed figure that looks like a V-letter of the unit cell and a strut of the linker portion, which is a line that forms the linker portion.

8. The polymeric stent of claim 7, wherein an area of the strut of the unit cell and the strut of the linker portion is 5 to 46%, based on an area of a rectangle which passes through both ends of the first hinge portion, both ends of the second hinge portion, and an end of the linker portion.

9. The polymeric stent of claim 7, wherein a thickness of the strut of the unit cell and the strut of the linker portion is 80 to 160 μm.

10. The polymeric stent of claim 1, wherein when the polymeric stent is a tube type, a diameter of the polymeric stent is 2 to 8 mm.

11. The polymeric stent of claim 1, wherein the polymeric stent comprises a biodegradable polymer.

12. The polymeric stent of claim 1, wherein the repeating unit is formed by processing a polymeric tube using laser.

13. A method for manufacturing a polymeric stent, comprising:
forming a repeating unit including a unit cell having a shape of a closed figure that looks like a V-letter and including a first hinge portion bent inwardly to the unit cell and a second hinge portion facing the first hinge portion and bent outwardly from the unit cell; and a linker portion extended outwardly from the bent portion of the second hinge portion; and
disposing the repeating units, such that an end of a linker portion of one repeating unit is connected to a bent portion of a first hinge portion of another adjacent repeating unit, or
forming the repeating units continuously such that an end of a first hinge portion and an end of a second hinge portion of one repeating unit are connected to an end of a first hinge portion and an end of a second hinge portion of the another adjacent repeating unit, respectively.

14. The method of claim 13, wherein the repeating unit is formed by using laser.

15. The method of claim 13, wherein the repeating unit is formed on a side surface of a polymeric tube.

16. The method of claim 14, wherein the repeating unit is formed on a side surface of a polymeric tube.
